(19) United States
(12) Patent Application Publication
(10) Pub. No.: US 2011/0280807 A1
(43) Pub. Date: Nov. 17, 2011

(54) Methods and Models for Stress-Induced Analgesia

(75) Inventor: Andrea G. Hohmann, Bloomington, IN (US)

(73) Assignee: UNIVERSITY OF GEORGIA RESEARCH FOUNDATION, INC., Athens, GA (US)

(21) Appl. No.: 11/912,755

(22) PCT Filed: Apr. 27, 2006
(86) PCT No.: PCT/US06/16296

§ 371 Date: May 13, 2011

Related U.S. Application Data
(60) Provisional application No. 60/676,532, filed on Apr. 28, 2005.

(51) Int. Cl. A61K 31/454; A61K 31/5383; A61K 31/55; A61K 49/00; A61K 31/353; A61K 31/4045; A61P 25/04; A61K 31/415; A61K 31/4525

(52) U.S. Cl. 424/9.2; 514/326; 514/406; 514/230.2; 514/213.01; 514/282; 514/454; 514/415

(57) ABSTRACT

The invention disclosed relates to methods and models for enhancing stress-induced analgesia through non-opioid mechanisms.

This application is a 35 USC 371 national state patent application derived from PCT International Patent Application No. PCT/US2006/016296 filed on 27 Apr. 2006, in the name of University of Georgia Research Foundation, Inc., a U.S. national corporation, applicant for the designation of all countries, and Andrea G. Hohmann, a citizen of the U.S., applicant for the designation of the US only, and claims priority to U.S. Provisional Application No. 60/676,532, filed Apr. 28, 2005, now abandoned.

### STATEMENT AS TO RIGHTS TO INVENTIONS MADE UNDER FEDERALLY SPONSORED RESEARCH AND DEVELOPMENT

This research was supported in part by grants from the National Institute on Drug Abuse DA14265 and DA014022. The US government may have certain rights in the invention.

### BACKGROUND OF THE INVENTION

Stress activates endogenous pain inhibitory systems in the brain that block pain through descending mechanisms, thus preventing the transmission of impulses from nociceptors to the central nervous system. This phenomenon, termed stress-induced analgesia, is mediated in part by the release of endogenous opioids. However, environmental stressors also activate endogenous mechanisms for suppressing pain that are insensitive to blockade by opioid antagonists such as naltrexone or naloxone. This classic demonstration provided early evidence for the existence of endogenous non-opioid analgesic systems. Although non-opioid stress analgesia was first described over twenty years ago and has been the subject of extensive investigation, the endogenous mediator(s) that produce these profound changes in pain sensitivity have remained unknown. See for example, Lewis et al, Science 208, 623-5 (1980), Science 217, 557-9 (1982), J Neurosci 1, 358-63 (1981); Grau et al, Science 213, 1409-11 (1981); Terman et al, Brain Res 260, 147-50 (1983), Science 226, 12707 (1984); Akil et al, Annals of the New York Academy of Sciences 467, 140-53 (1986); Maier et al, J Exp Psychol Anim Behav Process 9, 80-90 (1983); Valverde et al, Eur J Neurosci 12, 533-9 (2000).

The identification of cannabinoid receptors and endocannabinoids such as anandamide and 2-arachidonylglycerol established the existence of a cannabinoid transmitter system—an endogenous nonopioid system that acts through a marijuana-like mechanism. See for example, Matsuda et al, Nature 346, 561-4 (1990); Herkenham et al, J. Neurosci. 11, 563583 (1991); Devane et al, Science 258, 1946-9 (1992); Mechoulam et al, Biochem Pharmacol 50, 83-90 (1995); Sugiura et al, Biochem Biophys Res Commun 215, 89-97. (1995).

Behavioral, electrophysiological and neurochemical studies demonstrate that cannabinoids play a functional role in the nervous system to suppress pain. However, the environmental factors that activate the release of endocannabinoids are poorly understood. The distribution of cannabinoid receptors and FAAH, an endocannabinoid degrading enzyme, in brain areas related to stress provide anatomical support for the hypothesis that stressors produce analgesia independently of endogenous opioids via a cannabinoid mechanism. See, Calignano, et al, Nature 394, 277-281 (1998); Martin et al, J. Neurosci. 16, 6601-6611 (1996); Hohmann et al, Life Sci. 56, 2111-2118 (1995); Meng et al, Nature 395, 381-384 (1998); Walker et al, Prostaglandins Leukot Essent Fatty Acids 66, 235-42 (2002); Tsou et al, Neurosci Lett 254, 137-40 (1998); Egertová et al, Neuroscience 119, 481-96 (2003); Deutsch et al, Biochem Pharmacol 46, 791-6 (1993); Cravatt et al, Nature 384, 83-7 (1996).

The predominant methods for producing analgesia and treating pain and related disorders involves the use of compounds that act primarily via endogenous opioid systems. The use of such compounds, however, is problematic, often associated with the development of tolerance, dependence and abuse. Thus, there exists a need in the art for alternative methods of treating pain through non-opioid mechanisms, new animal models for studying the neurobiology of analgesia, as well as new models for identifying compounds that work through these mechanisms.

### SUMMARY OF THE INVENTION

In its broadest aspects, the present invention provides methods for: potentiating stress-induced analgesia; treating a stress-induced disorder or condition; enhancing or potentiating stress-induced analgesia through stimulation of central nervous system cannabinoid receptors; producing analgesia in a patient tolerant to morphine; and for testing or screening compounds that mediate non-opioid stress-induced analgesia.

### BRIEF DESCRIPTION OF THE DRAWINGS

FIG. 1: A CB1 mechanism mediates nonopioid stress-induced analgesia (SIA). a, SR141716A but not naltrexone or SR144528 blocks SIA. Inset: A significant overall drug effect (P<0.004) is shown. b, SR141716A failed to alter tail-flick latencies in the absence of footshock. c, The VR1 antagonist capsazepine failed to alter SIA. d, The CB1 antagonist AM251 suppressed SIA. e, SIA was attenuated in chronic WIN55,212-2 relative to vehicle or acute WIN55,212-2 groups (P<0.0002). Inset: Post-WIN55,212-2 tail-flick latencies were higher on day 2 compared to day 7 or 14 (P<0.0002). f, SIA did not differ in chronic morphine and vehicle groups. Inset: Post-morphine tail-flick latencies were higher on day 1 compared to day 7 (P<0.0002). Data are Mean±SEM. **P<0.01, *P<0.05 for all comparisons.

FIG. 2: Stress enhances Δ⁹-THC analgesia whereas Δ⁹-THC or inhibition of FAAH enhances stress-induced analgesia (SIA). a, Δ⁹-THC-induced antinociception was greater in rats subjected to footshock (S+THC) compared to non-shocked rats (NS+THC) (P<0.03; ^{x}P<0.05 different from Pre-drug baseline; *P<0.05 different from NS+THC). b, Δ⁹-THC potentiated SIA relative to vehicle (P<0.02). c, Potentiation of SIA by AA-5-HT is blocked by SR141716A. Inset: A significant overall drug effect (P<0.0002) is shown. Data are Mean±SEM. **P<0.01, *P<0.05 different from other groups, ^{##}P<0.01 different from AA-5-HT, ^{x}P<0.05 different from vehicle.

FIG. 3: The dorsolateral periaqueductal gray is implicated in cannabinoid stress-induced analgesia (SIA). Site-specific administration of (a,c) SR141716A (1 μg) suppressed and (b,d) AA-5-HT (AR, 10 μg) enhanced SIA. Inset: A significant overall drug effect (P<0.0002 for a,b) is shown. Data are Mean±SEM. ***P<0.0002; **P<0.01, *P<0.05. c, d, Reconstruction of microinjection sites for drug (closed symbols) and vehicle (open symbols) groups. Chromatograms show co-elution of (e) endogenous 2-AG in a dorsal midbrain sample with (f) synthetic 2-AG standard. g, 2-AG levels are elevated (t-test, P<0.04) in dorsal midbrain extracts derived from rats subjected to the stressor (S) relative to non-shocked control rats (NS).

### DETAILED DESCRIPTION OF THE INVENTION

This invention relates to our finding central nervous system cannabinoid as a novel therapeutic target for treating stress-related and pain-related disorders. Acute stress suppresses pain by activating brain pathways that engage both opioid and non-opioid mechanisms. Injection of CB1 cannabinoid receptor antagonists into the periaqueductal gray matter (PAG) of the midbrain can prevent non-opioid stress-induced analgesia.

Accordingly, the invention provides new methods for screening for or identifying compounds modulating stress-induced responses or conditions.

The practice of the present invention will employ, unless otherwise indicated, conventional methods of chemistry, biochemistry, molecular biology, cell biology, genetics, immunology and pharmacology, within the skill of the art. Such techniques are explained fully in the literature. See, e.g., Gennaro, A. R., ed. (1990) Remington's Pharmaceutical Sciences, 18th ed., Mack Publishing Co.; Hardman, J. G., Limbird, L. E., and Gilman, A. G., eds. (2001) The Pharmacological Basis of Therapeutics, 10th ed., McGraw-Hill Co.; Colowick, S. et al., eds., Methods In Enzymology, Academic Press, Inc.; Weir, D. M., and Blackwell, C. C., eds. (1986) Handbook of Experimental Immunology, Vols. I-IV, Blackwell Scientific Publications; Maniatis, T. et al., eds. (1989) Molecular Cloning: A Laboratory Manual, 2nd edition, Vols. I-II, Cold Spring Harbor Laboratory Press; Ausubel, F. M. et al., eds. (1999) Short Protocols in Molecular Biology, 4th edition, John Wiley & Sons; Ream et al., eds. (1998) Molecular Biology Techniques: An Intensive Laboratory Course, Academic Press; Newton, C. R., and Graham, A., eds. (1997) PCR (Introduction to Biotechniques Series), 2nd ed., Springer Verlag.

Unless defined otherwise, all technical and scientific terms used herein have the meaning commonly understood by a person skilled in the art to which this invention belongs. The following references provide one of skill with a general definition of many of the terms used in this invention: Singleton et al., DICTIONARY OF MICROBIOLOGY AND MOLECULAR BIOLOGY (2d ed. 1994); THE CAMBRIDGE DICTIONARY OF SCIENCE AND TECHNOLOGY (Walker ed., 1988); THE GLOSSARY OF GENETICS, 5TH ED., R. Rieger et al. (eds.), Springer Verlag (1991); and Hale & Marham, THE HARPER COLLINS DICTIONARY OF BIOLOGY (1991). As used herein, the following terms have the meanings ascribed to them unless specified otherwise.

It is noted here that as used in this specification and the appended claims, the singular forms “a,” “an,” and “the” include plural reference unless the context clearly dictates otherwise.

The term “composition”, as in pharmaceutical composition, is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier. The term “pharmaceutical composition” indicates a composition suitable for pharmaceutical use in a subject, including an animal or human. A pharmaceutical composition generally comprises an effective amount of an active agent and a pharmaceutically acceptable carrier.

The term “pharmaceutically acceptable carrier” encompasses any of the standard pharmaceutical carriers, buffers and excipients, including phosphate-buffered saline solution, water, and emulsions (such as an oil/water or water/oil emulsion), and various types of wetting agents and/or adjuvants. Suitable pharmaceutical carriers and their formulations are described in REMINGTON'S PHARMACEUTICAL SCIENCES (Mack Publishing Co., Easton, 19th ed. 1995). Preferred pharmaceutical carriers depend upon the intended mode of administration of the active agent. Typical modes of administration are described below.

The term “effective amount” means a dosage sufficient to produce a desired result on health, including, but not limited to, disease states. The desired result may comprise a subjective or objective improvement in the recipient of the dosage. A subjective improvement may be, for instance with respect to pain, decreased sensation of pain (e.g., non-inflammatory pain, neuropathic pain). An objective improvement may be, for instance, an increased ability to move or use (e.g., place weight upon) an affected limb or a longer period of uninterrupted sleep, or a behavioral response indicating an increased tolerance of a painful stimuli.

A “prophylactic treatment” is a treatment administered to a subject who does not have the subject condition (e.g., pain), wherein the treatment is administered for the purpose of decreasing the risk of developing the condition or to counter the severity of the condition (e.g., anxiety; depression; pain, including but not limited to, acute pain, chronic pain, inflammatory pain, non-inflammatory pain, neuropathic pain and pain expected to result from the expected or likely occurrence of a painful event (e.g., surgery)) if one were to develop.

A “therapeutic treatment” is a treatment administered to a subject who has the condition (e.g., pain, and/or exhibits signs or symptoms of pain including but not limited to, acute pain, chronic pain, inflammatory pain, non-inflammatory pain, neuropathic pain, wherein treatment is administered for the purpose of diminishing or eliminating those signs or symptoms).

A “therapeutically effective amount” is an amount of an agent sufficient to reduce the signs and/or symptoms of the disease or condition or to prevent, oppose, or reduce their progression.

The term “modulate” means to induce any change including increasing and decreasing. A modulator of a receptor includes both agonists and antagonists of the receptor.

The term “treating” means combating, reducing, shortening, alleviating or eliminating a condition of the subject (e.g., pain, anxiety, or depression). Pain, particularly severe pain, can be a stressor. Thus, in one aspect the invention is drawn to methods of treating chronic pain conditions, including neuropathic pain, and chronic or intermittent pain associated with chronic health conditions as such conditions are often substantial stressors. “Neuropathic pain” is pain caused by a primary lesion or dysfunction of the nervous system. Such pain is chronic and involves a maintained abnormal state of increased pain sensation, in which a reduction of pain threshold and the like are continued, due to persistent functional abnormalities ensuing from an injury or degeneration of a nerve, plexus or perineural soft tissue. Such injury or degeneration may be caused by wound, compression, infection, cancer, ischemia, or a metabolic or nutritional disorder such as diabetes mellitus. Neuropathic pain includes, but is not limited to, neuropathic allodynia wherein a pain sensation is induced by mechanical, thermal or another stimulus that does not normally provoke pain, neuropathic hyperalgesia wherein an excessive pain occurs in response to a stimulus that is normally less painful than experienced. Examples of neuropathic pain include diabetic polyneuropathy, entrapment neuropathy, phantom pain, thalamic pain after stroke, post-herpetic neuralgia, atypical facial neuralgia pain after tooth extraction and the like, spinal cord injury, trigeminal neuralgia and cancer pain resistant to narcotic analgesics such as morphine. The neuropathic pain includes the pain caused by either central or peripheral nerve damage. And it includes the pain caused by either mononeuropathy or polyneuropathy (e.g., familial arnyloid polyneuropathy). As compared to inflammatory pain, neuropathic pain is relatively resistant to therapy with nonsteroidal anti-inflammatory agents and opioid substances (e.g., morphine).

Neuropathic pain may be bilateral in mirror image sites, or may be distributed approximately according to the innervation of the injured nerve, it may persist for months or years, and be experienced as a burning, stabbing, shooting, throbbing, piercing electric shock, or other unpleasant sensation.

The subject species to which the treatments can be given according to the invention are mammals, and include, but are not limited to, humans, primates, rodents, rats, mice, rabbits, horses, dogs and cats. In preferred embodiments of each aspect, the subject is human.

Peroxisome proliferator activated receptors (PPAR) are a family of transcription factors and have been principally studied with respect to lipid homeostasis. Three PPAR subtypes have been identified: α, β (also described as δ), and γ. All three subtypes have domain structure common with other members of the nuclear receptor family. DNA-binding domains are highly conserved among PPAR subtypes, but ligand binding domains are less well conserved. (Willson, et al. (2000) J Med Chem. 43:527). PPARs bind to RXR transcription factors to form heterodimers that bind to DNA sequences containing AGGTCAnAGGTCA. It has been shown that ligand binding to PPAR can induce gene expression. PPARα has been reported to inhibit inflammatory edema and inflammatory pain (see Taylor et al. Inflammation 26(3):121 (2002) and Sheu et al. J. Invest. Dermatol. 118:94 (2002)). Suitable PPARα agonists, CBI receptor agonists, and FAAH inhibitors, and anandamide transport inhibitors for use according to the present invention are disclosed in U.S. Provisional Patent Application No. 60/565,196, fled Apr. 23, 2004 and assigned to the same assignee as the present application, and incorporated by reference herein in its entirety and particularly with respect to the PPARα agonist (e.g., PPARα activator, partial agonist, full agonist), CB₁receptor agonist, FAAH inhibitor, and anandamide transport inhibitor subject matter disclosed therein.

### CB1 Receptor Agonists for Use According to the Invention.

A variety of CB1 receptor agonists are known to date; these include classical cannabinoids, such as, for example, Δ⁹-THC, non-classical cannabinoids, aminoalkylindoles and eicosanoids. The latter include the generally accepted endogenous CB1 receptor agonist anandamide. In all such above aspects of the invention, and embodiments thereof, setting forth a CB 1 cannabinoid receptor agonist, in a further embodiment thereof the CB 1 cannabinoid receptor agonist is CP-55940, Win-55212-2, anandamide, methanandamide, or 2-arachidonoylglycerol.

CB1 Receptor Agonists for use according to the invention, include but are not limited to, compounds as taught in U.S. Pat. No. 5,631,297.

In accordance with this aspect of the present invention, there are disclosed pharmaceutical compositions and methods for treating pain comprising use of direct acting cannabinoid receptor agonists (e.g., arachidonylethanolamide (anandamide), (R)-(+)arachidonyl-1-hydroxy-2-propylamide, cis-7,10,13,16-docosatetraenoylethanolamide, homo-delta-linoleyethanolamide, N-propyl-arachidonylethanolamide, N-ethyl-arachidonylethanolamide, and 2-arachidonylglycerol, and indirect acting FAAH inhibitors N-(4-hydroxyphenyl)-arachidonylamide, palmitylsulphonylfluoride, and arachidonyltrifluoromethylketone.

### Cannabinoid Receptor Activity Screening.

While a great many CB1 agonist compounds are known in the art, additional suitable novel CB1 agonist compounds can be readily identified using methods known in the art. For instance, methods for screening compounds for CB1 agonist activity are well known to one of ordinary skill in the art. A variety of means may be used to screen cannabinoid CB1 receptor activity in order to identify the compounds for use according to the invention. A variety of such methods are taught in U.S. Pat. No. 5,747,524 and U.S. Pat. No. 6,017,919.

### Ligand Binding Assays.

Ligand binding assays are well known to one of ordinary skill in the ar. For instance, see, U.S. Patent Application No. US 2001/0053788 published on Dec. 20, 2001, U.S. Pat. No. 5,747,524, and U.S. Pat. No. 5,596,106 and (see, Felder, et al., Proc. Natl. Acad Su., 90:7656-7660 (1993)) each of which is incorporated herein by reference. The affinity of an agent for cannabinoid CB 1 receptors can be determined using membrane preparations of Chinese hamster ovary (CHO) cells in which the human cannabis CB1 receptor is stably transfected in conjunction with [³H]CP-55,940 as radioligand. After incubation of a freshly prepared cell membrane preparation with the [³H]-ligand, with or without addition of compounds of the invention, separation of bound and free ligand can be performed by filtration over glassfiber filters. Radioactivity on the filter was measured by liquid scintillation counting.

The cannabinoid CB1 agonistic activity of a candidate compound for use according to the invention can also be determined by functional studies using CHO cells in which human cannabinoid CB 1 receptors are stably expressed. Adenylyl cyclase can be stimulated using forskolin and measured by quantifying the amount of accumulated cyclic AMP. Concomitant activation of CB1 receptors by CBI receptor agonists (e.g., CP-55,940 or (R) WIN-55,212-2) can attenuate the forskolin-induced accumulation of cAMP in a concentration-dependent manner. This CB1 receptor-mediated response can be antagonized by CB1 receptor antagonists. See, U.S. Patent Application No. US 2001/0053788 published on Dec. 20, 2001.

Samples rich in cannabinoid CB1 receptors and CB2 receptors, rat cerebellar membrane fraction and spleen cells can be respectively used (male SD rats, 7-9 weeks old). A sample (cerebellar membrane fraction: 50 μ·g/ml or spleen cells: 1(×10⁷ cells/ml), labeled ligand ([³ H]Win55212-2, 2 nM) and unlabeled Win55212-2 or a test compound can be plated in round bottom 24 well plates, and incubated at 30° C. for 90 min in the case of cerebellar membrane fraction, and at 4° C. for 360 min in the case of spleen cells. As the assay buffer, 50 mM Tris solution containing 0.2% BSA can be used for cerebellar membrane fraction, and 50 mM Tris-HBSS containing 0.2% BSA can be used for spleen cells. After incubation, the samples are filtrated through a filter (Packard, Unifilter 24 GF/B) and dried. A scintillation solution (Packard, Microsint-20) can be added, and the radioactivity of the samples determined (Packard, Top count A9912V). The non-specific binding can be determined by adding an excess Win55212-2 (1 μM), and calculating specific binding by subtracting non-specific binding from the total binding obtained by adding the labeled ligand alone. The test compounds can be dissolved in DMSO to the final concentration of DMSO of 0.1%. EC₅₀ can be determined from the proportion of the specifically-bound test compounds, and the Kᵢ value of the test compounds can be calculated from EC₅₀ and K_{d} value of [³H]WIN55212-2. See, U.S. Pat. No. 6,017,919.

In one embodiment, the EC₅₀ for cannabinoid receptor binding is determined according to the method of Devane, et al., Science, 258: 1946-1949 (1992) and Devane, et al., J. Med. Chem., 35:2065 (1992). In this method, the ability of a compound to competitively inhibit the binding of a radiolabeled probe (e.g., ³H-HU-2430) is determined.

In other embodiments, the EC₅₀ of an agonist for the CB1 receptor is determined according to any one of the above ligand binding assay methods. In another embodiment, the EC₅₀ is according to any assay method which studies binding at physiological pH or physiologically relevant conditions. In another embodiment, the EC₅₀ is determined according to any assay method which studies binding at physiological pH and ionic strength. Preferred assay incubation temperatures range from 20° C.-37° C. Temperatures may be lower or higher. For instance, incubation temperatures of just a few degrees or 0° C. may be useful in preventing or slowing the degradation of enzymatically unstable ligands. Inhibitors of FAAH may also be added to protect antagonists from degradation.

Cannabinoid CB2 Receptor Binding Assay.

A variety of means may be used to screen cannabinoid CB2 receptor activity in order to identify compounds for use according to the invention. Methods of studying CB2 receptor binding are well known to one of ordinary skill in the art. For instance, binding to the human cannabinoid CB2 receptor can be assessed using the procedure of Showalter, et al., J. Pharmacol Exp Ther., 278(3):989-99 (1996)), with minor modifications as taught for instance in U.S. Patent Application No. 20020026050, published Feb. 28, 2002. Each of which is incorporated herein by reference.

In other embodiments, the EC₅₀ of an inventive compound for the CB2 receptor is determined according to any one of the above CB2 receptor ligand binding assay methods. In another embodiment, the EC₅₀ is according to any assay method which studies binding at physiological pH or physiologically relevant conditions. In another embodiment, the EC₅₀ is determined according to any assay method which studies binding at physiological pH and ionic strength. Preferred assay incubation temperatures range from 20° C.-37° C. Temperatures may be lower or higher. For instance, incubation temperatures of just a few degrees or 0° C. may be useful in preventing or slowing the degradation of enzymatically unstable ligands. Inhibitors of FAAH may also be added to protect antagonists from degradation.

### Methods for Assessing Ability of a Compound to Modulate Stress-Induced Responses Stress-induced Analgesia or Pain-Relief.

Methods for screening compounds for an antinociceptive effect are well known to one of ordinary in the art. For instance, the test compounds can be administered to the subject animals in the mouse hot-plate test (Beltramo et al., Science, 277:1094-1097 (1997)) and the mouse formalin test and the nociceptive reactions to thermal or chemical tissue damage measured. See also U.S. Pat. No. 6,326,156 which teaches methods of screening for antinociceptive activity. See Cravatt et al. Proc. Natl. Acad Sci. USA. 98:9371-9376 (2001). A method of testing for antinociception is set forth in the Examples.

A fully automatic tail-flick analgesiameter (IITC Model 336; Woodland Hills, Calif.) may be used to assess tail-flick latencies. This assessment of tail-flick latency is not subject to bias. Removal of the tail from the radiant heat source is initiated by the rat, which automatically terminates the heat stimulus. The tail-flick latency is calculated by the electronic analgesia meter without intervention of the experimenter. Tail-flick latencies can be assessed in a manner identical to that described in the art (Walker et al. PNAS 96, 12198 12203, 1999; Martin et al. JNsci 16, 6601-6611, 1996).

The diagnosis and assessment of neuropathic pain is well known to one of ordinary skill in the art. Pain can be identified and assessed according to its onset and duration, location and distribution, quality and intensity, and secondary signs and symptoms (e.g., mood, emotional distress, physical or social functioning), and triggering stimulus or lack thereof. For human subject, often subjective pain assessment scales are used to measure intensity. Such scales may grade pain intensity verbally ranging from no pain-mild pain moderate pain-severe pain-very severe pain and worst possible pain, or on a numeric scale from 1 (no pain) to 5 (moderate pain) to 10 (worst possible pain).

Suitable animal models for testing the ability of agents to treat neuropathic pain are also known to one of ordinary skill in the art. Such methods have been the subject of recent review (Wang et al. Advanced Drug Delivery Reviews 55:949 (2003)) which is incorporated by reference herein in its entirety. Methods of assessing neuropathic pain include 1) the weight drop or contusion model of Allen; 2) the photochemical SCI model; 3) the excitotoxic spinal cord injury model; 4) the neuroma model; 5) the chronic constriction injury model of Bennett; 6) the partial sciatic nerve ligation model; 7) the L5/L6 spinal ligation model; 8) the sciatic cryoneurolysis model; and 9) the sciatic inflammatory neuritis model. In addition there are a variety of models for studying the neuropathic pain of diabetes polyneuropathy; toxic neuropathies; and various bone cancer models.

### Pain.

As pain is a stressor itself, in some embodiments, the compounds, compositions, and methods of treatment according to the invention are administered to alleviate pain in a subject. One or ordinary skill in the art can identify severe pain conditions or stressful conditions likely to induce stress-induce analgesia. The treatment may be prophylactic or therapeutic. The treatment may be administered to a human subject in need of pain relief or modulation of stress-induced analgesia. The compounds and compositions of the invention may be administered solely for the purposes of reducing the severity or frequency or extent of pain. The treatment may be administered in a combination therapy with another pain reliever or an anti-inflammatory agent.

Pain, in particular, can be a stressor, and also a condition subject to treatment according to the invention. Thus, in one aspect the invention is drawn to methods of treating chronic pain conditions, including neuropathic pain, and chronic or intermittent pain associated with chronic health conditions as such conditions are often substantial stressors. In other embodiments, the pain can be a neuropathic pain.

The pharmaceutically active agents (e.g., FAAH inhibitors, MGL inhibitors, COX-2 inhibitors, cannabinoid receptor agonists, opioids, NSAIDs, anandamide transport inhibitors, and PARa agonists) to be used according to the invention may be administered by a variety of routes. These routes include, but are not limited to, the oral route, the intravenous route, and the dermal routes of administration. They may be administered locally (e.g., near the site of the pain or the primary lesion or dysfunction) or systemically. When one or more active agents are to be administered, they may be administered concurrently or at different times. They may be administered on the same or different schedules (e.g., according to the biological half-times in the body or their individual duration of action). They may be administered together via one pharmaceutical composition or via separate pharmaceutical compositions.

The following examples are provided to illustrate, and not to limit, the invention.

### EXAMPLES

The existence of a cannabinoid mechanism of non-opioid stress-induced analgesia is demonstrated by: 1) blocking cannabinoid CB1 receptors, 2) rendering rats tolerant to the antinociceptive effects of cannabinoids, 3) inhibiting the enzymatic breakdown of endocannabinoids, 4) identifying the periaqueductal gray as a primary site of action, and 5) quantifying stress-induced changes in levels of endocannabinoids using high performance liquid chromatography tandem mass spectrometry (LC/MS/MS).

### Example 1

### Role of Cannabinoids in Nonopioid Stress Analgesia

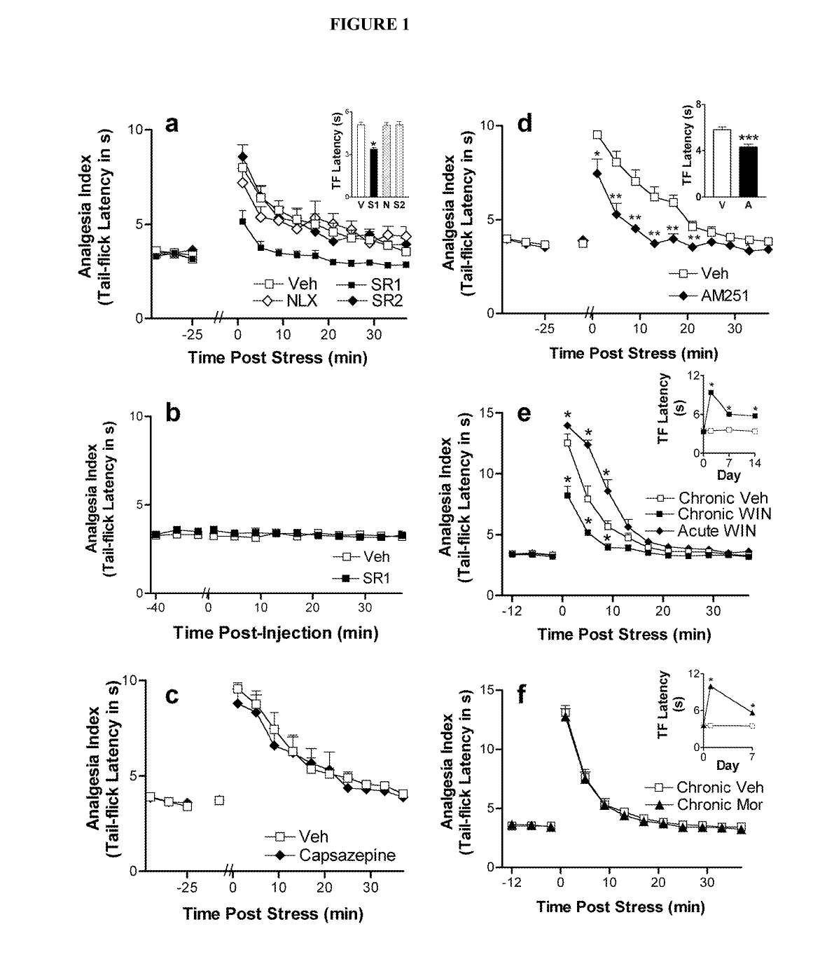

To evaluate the role of cannabinoids in nonopioid stress analgesia, rats were subjected to brief, continuous footshock using the paradigm established by Lewis (Lewis et al, Science 208, 623-5 (1980). Stress analgesia was quantified using the tail-flick test. Antagonist Studies. After establishing baseline tail-flick latencies, SR1417161A (5 mg/kg; n=8), SR144258 (5 mg/kg; n=8), naltrexone (14 mg/kg; n=6) or vehicle (E:E:S; n=11) (FIG. 1a), AM251 (5 mg/kg; n=12) or vehicle (DMSO; n=12) (FIG. 1d) or capsazepine (10 mg/kg; n=6) or vehicle (n=6) (FIG. 1c) was administered intraperitoneally 20 min prior to footshock. Tail-flick latencies were also measured in groups receiving SR141716A (n=6) or vehicle (n=6) in the absence of the stressor (FIG. 1b) at the same times.

Stress analgesia was markedly attenuated in rats receiving SR141716A, a competitive antagonist/inverse agonist for central cannabinoid CB1 receptors (Rinaldi-Carmona et al, FEBS Lett. 350, 240-244 (1994), relative to vehicle (FIG. 1a). By contrast, neither the opioid antagonist naltrexone (14 mg/kg i.p.) nor the CB2 antagonist SR144528 altered stress analgesia in this paradigm (FIG. 1a). Prior to administration of the stressor, baseline responses to thermal stimulation of the tail did not differ between groups.

The SR141716A-induced suppression of stress analgesia cannot be attributed to a nonspecific change in basal nociceptive thresholds; in the absence of the stressor, SR141716A failed to alter tail-flick latencies (FIG. 1b). The effects of SR141716A are mediated by CB1; although SR141716A inhibits vanilloid VR1 receptors at high concentrations and anandamide binds to VR1 with low affinity (De Petrocellis et al FEBS Lett 483, 52-6 (2000); Zygmunt et al, Nature 400, 452-7 (1999)), the VR1 antagonist capsazepine (10 mg/kg i.p.) did not alter stress analgesia in this paradigm (FIG. 1c). Moreover, a pharmacologically distinct CB1 antagonist, AM251, also blocked this stress analgesia in the absence of changes in basal nociceptive thresholds (FIG. 1d). These data indicate that stress-induced analgesia elicited by brief continuous footshock is selectively mediated by a cannabinoid CB1 mechanism.

### Example 2

Pharmacological Specificity of Cannabinoid-Mediated Stress Analgesia

To further evaluate the pharmacological specificity of cannabinoid-mediated stress analgesia, we tested the hypothesis that tolerance to cannabinoids would attenuate nonopioid stress analgesia. Tolerance Studies. Rats received repeated daily injections (i.p.) of vehicle (n=10), WIN55,212-2 (10 mg/kg×14 days; n=11) or WIN55,212-2 administered acutely (10 mg/kg on day 14; n=8). Post-injection tail-flick latencies were measured on days 2, 7 and 14 to confirm that the injection paradigm induced tolerance to the antinociceptive effects of cannabinoids before administration of the stressor (FIG. 1d Inset). Morphine antinociception (2.5 mg/kg s.c. on day 15) was compared in separate groups treated chronically with WIN55,212-2 (n=7) or vehicle (n=6) in lieu of exposure to the stressor. Separate groups received daily injections of vehicle (n=8) or morphine (10 mg/kg s.c.×7 days; n=8). Post-injection tail-flick latencies were measured on days 1 and 7 to confirm that rats were tolerant to the antinociceptive effects of morphine (FIG. 1e Inset). Twenty-four h following the terminal injection, tail-flick latencies were assessed, rats were subjected to footshock, and stress analgesia was quantified over 60 min (FIG. 1e-f). Ceiling tail-flick latencies were 14 s to permit detection of enhancements of stress analgesia following acute drug exposure.

Repeated daily injections of WIN55,212-2 induced tolerance to cannabinoid antinociception prior to administration of the stressor (FIG. 1e inset). Stress analgesia was attenuated in rats rendered tolerant to the antinociceptive effects of the cannabinoid (FIG. 1e); this attenuation was apparent when the cannabinoid-tolerant animals were compared with rats treated chronically with vehicle or acutely with WIN55,212-2. By contrast, just prior to administration of the stressor, baseline tail-flick latencies did not differ between groups. In addition, stress analgesia was increased in groups treated acutely with the cannabinoid (24 h prior to footshock) relative to vehicle (FIG. 1e). These data suggest that acute exposure to a cannabinoid rendered rats hypersensitive to the analgesic effects of stress. To confirm that tolerance-induced changes in nonopioid stress analgesia were not mediated by putative regulatory changes in μ-opioid responsive systems, we examined the antinociceptive effects of morphine (2.5 mg/kg s.c. on day 15) in rats treated chronically with either WIN55,212-2 or vehicle in lieu of exposure to the stressor. No deficits in morphine analgesia were observed in rats rendered tolerant to the cannabinoid; in fact, a modest enhancement in morphine analgesia was observed in the cannabinoid-tolerant group relative to controls (post-morphine tail-flick latencies Mean±SEM: 6.2±0.25 vs. 5.3±0.21 s in rats treated chronically with WIN55,212-2 (n=7) and vehicle (n=6), respectively; F1,11 =6.90 P<0.03).

To confirm the specificity of the cross-tolerance of cannabinoid and nonopioid stress analgesia, we rendered rats tolerant to the antinociceptive effects of morphine. No differences in stress analgesia were observed between groups treated chronically with either morphine or vehicle (FIG. 1f, inset) [see also Terman et al, Brain Res 368, 101-6 (1986)]. These data demonstrate that the attenuation of stress-analgesia observed in the cannabinoid tolerant rats cannot be accounted for by downstream regulatory changes in μ-opioid tone following the tolerance induction paradigm.

### Example 3

### Acute Stress Induces Hypersensitivity to the Antinociceptive Effects of Cannabinoids

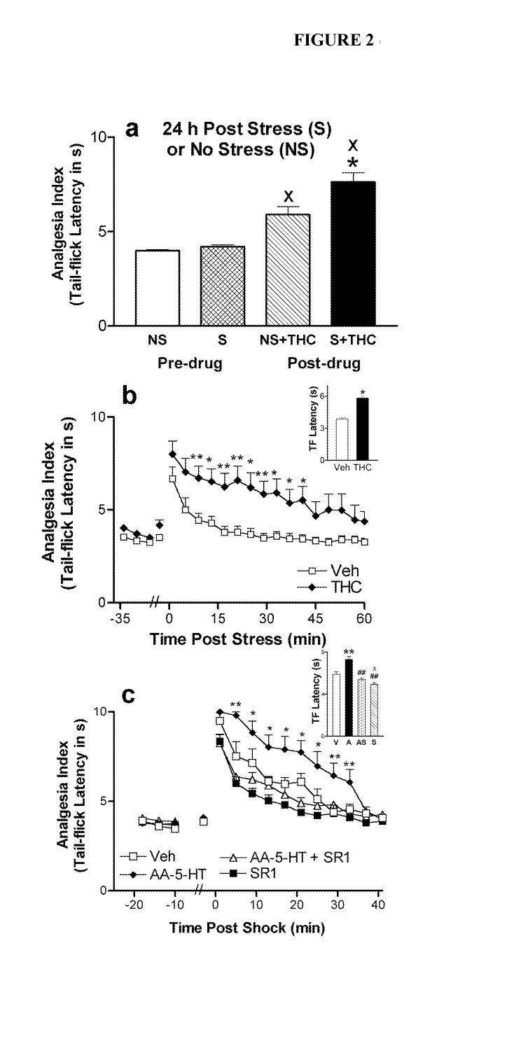

The prototypic cannabinoid Δ⁹-tetrahydrocannabinol (Δ⁹-THC) was used to test the hypothesis that rats subjected acutely to the stressor would be hypersensitive to the antinociceptive effects of cannabinoids. Δ⁹-THC Sensitization Studies. Tail-flick latencies were assessed prior to stress or no stress (home cage for 3 min) treatment (day 1) and Δ⁹-THC administration (day 2). On day 1, tail-flick latencies were measured 3 times at 2-min intervals following footshock (or no shock) treatment. On day 2 (FIG. 2a), Δ⁹-THC (10 mg/kg i.p.) was administered 24 h following stress (n=11) or no stress treatment (n=12). Tail-flick latencies were measured at 2-min intervals 28-32 min following A -THC administration. In a separate study (FIG. 2b), baseline tail-flick latencies were assessed prior to administration of Δ⁹THC (10 mg/kg i.p.) or vehicle (n=8). Tail-flick latencies were assessed 20 min following drug or vehicle administration immediately prior to footshock. Post-shock tail-flick latencies were measured over 60 min.

FAAH Inhibition Study. After establishing baseline tail-flick latencies, rats received (i.p.) AA-5-HT (10 mg/kg; n=7), vehicle (n=7), SR14176A (1 mg/kg; n=8) ten min prior to vehicle or SR141716A ten min prior to AA-5-HT (n=8). Tail-flick latencies were measured three times at 2-min intervals immediately prior to administration of the stressor (FIG. 2c). Rats were subjected to footshock 65 min following systemic administration of drug or vehicle, respectively.

Δ⁹-THC-induced antinociception was greater in rats subjected previously to the stressor compared to non-shocked control rats receiving the same dose (FIG. 2a). Moreover, Δ⁹-THC, administered prior to the stressor, enhanced the magnitude and the duration of nonopioid stress analgesia (FIG. 2b).

A potent and selective competitive inhibitor of FAAH, arachidonoylserotonin (Bisogno et al, Biochemical and biophysical research communications 248, 515-22 (1998); AA-5-HT), was used to test the hypothesis that blocking an enzyme that inactivates the endocannabinoids anandamide and 2-AG in vitro would enhance cannabinoid-mediated stress analgesia. AA-5-HT increased the magnitude and duration of cannabinoid stress analgesia. The effects of AA-5-HT were blocked by SR141716A, suggesting that inhibition of FAAH enhanced cannabinoid stress analgesia though a CB1 mechanism. These data are consistent with the observation that mice lacking FAAH are impaired in their ability to degrade endocannabinoids and exhibit profound CB1-dependent analgesia when treated with exogenous anandamide (Cravatt et al, Proc Natl Acad Sci USA 98, 9371-6 (2001). We observed a similar CB1-mediated enhancement of nonopioid stress analgesia following administration of palmitoyltrifluoromethylketone, a potent inhibitor of FAAH and phospholipase A2 activity, and the putative ‘anandamide transport inhibitor’ AM404 (data not shown) that also induces inhibits FAAH (Glaser et al. Proc Natl Acad Sci USA 100, 4269-74 (2003)). These data collectively suggest that increasing the bioavailability of endocannabinoids that are degraded by FAAH (e.g., anandamide and 2-AG; Di Marzo et al Biochemical Journal 331, 15-9 (1998); Goparaju et al FEBS Letters 422, 69-73 (1998)) enhances cannabinoid-mediated stress analgesia.

### Example 4

### Evaluation of the Site of Action of Canabinoid Mediated Stress-Induced Analgesia

To evaluate the site of action, two separate pharmacological approaches were used to bidirectionally manipulate cannabinoid-mediated stress analgesia.

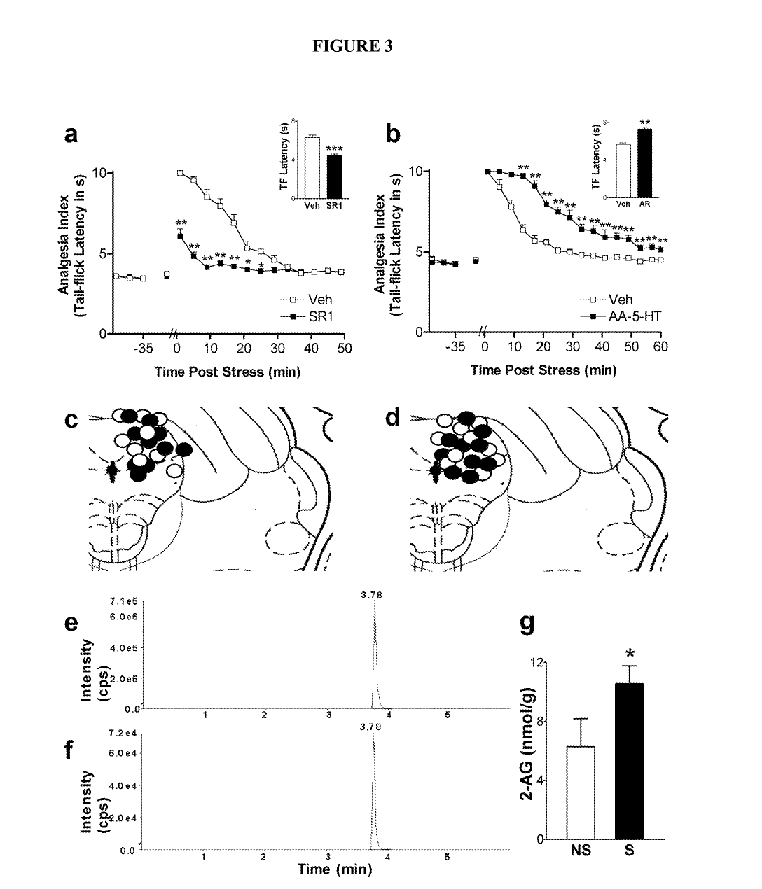

PAG Injection Studies. Stainless steel guide cannulae were implanted into the dorsolateral periaqueductal gray under pentobarbital/ketamine anesthesia 3-7 days prior to testing. After establishing baseline tail-flick latencies, rats received intracranial injections. Rats were subjected to footshock 5 min following microinjection of SR141716A (n=10) or vehicle (n=9; FIG. 3a) or 35 min following microinjection of AA5-HT (n=8) or vehicle (n=11; FIG. 3b). Tail-flick latencies were measured immediately prior to administration of the stressor and over 60 min post shock. Rats were perfused and cannulae placements verified microscopically (FIG. 3c-d).

Liquid Chromatography Tandem Mass Spectrometry (LC/MS/MS). Rats were habituated to the guillotine for 14 days prior to sacrifice. Rats were killed following exposure to footshock (3 min) or their home cages for the same times. Tissue from shocked (n=12) and nonshocked (n=12) rats was rapidly extracted, frozen in isopentane and stored (−80° C.) until use. D8-anandamide internal standards were added to samples in ice-cold methanol and sonicated. 2-AG and anandamide were isolated from methanol-extracts of samples by solid phase extraction on Bond Elut (Varian) C18 columns. Endocannabinoids were quantified (FIG. 3e-g) using isotope-dilution liquid chromatography tandem mass spectrometry (MDS Sciex/Applied Biosystems API3000). We examined, in positive ion multiple reaction monitoring mode, the transition from the precursor [M+H]+ ion 379.3 to 287.3 for 2-AG and [M+H]+ ion 348.3 to 287.3 for anandamide.

Administration of the CB1 antagonist SR141716A (1 μg) into the dorsolateral periaqueductal gray blocked nonopioid stress analgesia (FIG. 3a,c). Furthermore, inhibiting FAAH at the level of the dorsolateral periaqueductal gray with site-specific administration of AA-5-HT (10 μg) enhanced nonopioid stress analgesia (FIG. 3b,d). The actions of SR141716A and AA-5HT, respectively, were observed in the absence of changes in basal nociceptive thresholds determined immediately prior to administration of the stressor.

From the findings discussed above, it appears that exposure to the stressor activated an endocannabinoid analgesic mechanism. We used high-performance liquid chromatography tandem mass spectrometry to identify the endocannabinoid(s) mediating stress-induced analgesia. Levels of the endocannabinoid 2arachidonylglycerol (2-AG) were elevated (by 40.6%) in dorsal midbrain samples containing the dorsolateral periqueductal gray of rats subjected to the stressor relative to non-shocked control rats (FIG. 3c-e). Furthermore, levels of 2-AG in occipital cortex did not differ between groups (Mean±SEM [Shocked vs. Nonshocked]: 23.09±3.73 vs. 24.79±4.64 nmol/g). These data are consistent with the blockade and enhancement of stress analgesia observed in our behavioral studies following site-specific administration of SR141716A and AA-5-HT, respectively (FIG. 3a, b). Levels of anandamide in each region were similar between groups (Shocked vs. Nonshocked: 2.18±0.24 vs. 2.34±0.54 in dorsal midbrain; 10.31±1.73 vs. 9.66±1.19 pmol/g in occipital cortex)). Of course, stress-induced changes in levels of fatty acid amides are likely to be underestimated in the present work due to rapid metabolism of these mediators in vivo (Deutsch et al Biochem Pharmacol 46, 791-6 (1993); Cravatt et al Nature 384, 83-7 (1996)). Our data do not preclude the possibility that enzymes other than FAAH may be the predominant mechanism for degrading 2-AG (observed in the absence of stress) or that arachidonic acid metabolites formed in vivo subsequent to administration of exogenous 2-AG mediate effects independent of FAAH (Dinh, et al. Proc Nat Acad Sci 99, 10819-24 (2002); Chemistry and Physics of Lipids 121, 149-58 (2002); Lichtman et al, JPET 302, 73-9 (2002)).

The experiments described here demonstrate that specific types of stress activate an endogenous cannabinoid analgesic system, and that this effect occurs while endogenous opioid analgesic pathways remain quiescent. Hence, this endocannabinoidmediated stress-induced analgesia can be dissociated from endogenous opioid-mediated stress-induced analgesia. By contrast, opioid forms of stress-induced analgesia may rely in part upon CB 1, suggesting that endocannabinoids are the mainstay of endogenous stress-mediated analgesic phenomena. The endocannabinoid-mediated analgesic effects of stress likely represent only one of a constellation of physiological and behavioral changes produced by stress-induced activation of endocannabinoids. It will be of considerable interest to determine which of the other effects of stress, including those on affect, memory (Marsicano et al, Nature 418, 530-4. (2002)), hormonal regulation, and defensive aggression are also mediated by endocannabinoids. The finding that stress increases the bioavailability of endocannabinoids raises the possibility that endocannabinoid mechanisms represent a novel therapeutic target for treatment of stress-related disorders. The present observation of a behavioral hypersensitivity to the effects of Δ⁹-THC following acute exposure to a stressor may also suggest a neurochemical basis for vulnerability to marijuana abuse under conditions of environmental stress.

### Animals.

Adult male Sprague-Dawley rats were used for in vivo experiments and Wistar rats for enzymes assays and tissue cultures. All procedures were approved by the institutional animal care and use committee and followed guidelines of the International Association for the Study of Pain.

### Brain Slice Cultures.

Brain slices were cultured from Wistar rats. Pups were sacrificed on post-natal day 5 by decapitation following cryo-anaesthesia. Brains were removed and cut (0.4 mm-thick coronal slices) using a vibratome in a bath of ice-cold high glucose Dulbecco's Modified Eagle's Medium (Gibco). Hemispheres were placed on Millicell culture inserts (Millipore) in 6-well plates with serum-based culture medium (1.5 ml) composed of basal Eagle medium with Earle's salts (100 ml), Earle's balanced salt solution (50 ml), heat-inactivated horse serum (50 ml), L-glutamine (0.2 mM, 1 ml) and 50% glucose (2 ml) (Gibco). Slices were maintained at 37° C. with 5% CO2 for 7 days before use.

### Drugs.

Chemicals were from NIDA (SR141716A, SR144528), Sigma-Aldrich (WIN55,212-2, capsazepine, morphine sulfate, Δ⁹-THC), Cayman (arachidonoylserotonin, D8-anandamide) and Tocris (AM251). Drugs were dissolved in emulphor:ethanol:saline (E:E:S 1:1:8) or DMSO. Quantification of Stress Analgesia. Brief, continuous foot-shock (0.9 mA, AC current, 3 min) was administered to rats using a Lafayette gridshock apparatus. Withdrawal latencies to thermal stimulation of the tail were measured at 2-min intervals before and after footshock and calculated for each subject in 2-trial blocks. Ceiling tail-flick latencies were 10 s except where noted.

### Enzyme Assays.

Cell fractions were prepared from Wistar rat brain homogenates, and assayed cytosol MGL activity and membrane FAAH activity using 2-monooleoylglycerol[glycerol-1,2,3-³H] (ARC, St. Louis, Mo., 20 Ci/mmol), and anandamide[ethanolamine-³H] (ARC, St. Louis, Mo.), 60 Ci/mmol) respectively, as substrates^{23,24}.

### Surgery.

Stainless-steel guide cannulae were implanted in the left lateral ventricle or PAG (dorsolateral or ventrolateral), under pentobarbital/ketamine anaesthesia 3-7 days prior to testing. Cannulae placements were verified in Nissl-stained sections or by post mortem injection of Fast-green dye. Analyses were restricted to animals exhibiting dye spread throughout the ventricular system.

Tolerance Induction. Sprague-Dawley rats received daily i.p. injections of vehicle or WIN55212-2 for 2 weeks (10 mg-kg⁻¹ once daily). Morphine antinociception (2.5 mg-kg⁻¹ s.c. on day 15) was assessed in separate groups treated chronically with WIN55212-2 or vehicle. Separate groups received subcutaneous (s.c.) injections of vehicle or morphine (10 mg-kg⁻¹ once daily for 7 days). Post-injection tail-flick latencies were measured on days 2, 7 and 14 (chronic WIN55212-2 study) or days 1 and 7 (chronic morphine study) to confirm that the injection paradigm induced tolerance to the antinociceptive effects of each agonist prior to administration of the stressor. 24 h after the last injection, rats were subjected to foot shock, and stress analgesia was quantified. Ceiling tail-flick latencies were 15 s.

### Analgesia Tests.

Foot shock (0.9 mA, AC current, 3 min) was administered to Sprague-Dawley rats using a Lafayette grid-shock apparatus. Withdrawal latencies in the radiant heat tail-flick test^{11.17} were measured at 2-min intervals before (baseline) and after foot shock, and calculated for each subject in 2-trial blocks. Removal of the tail from the heat source automatically terminated application of thermal stimulation. Tail-flick latencies were monitored over 4 min immediately prior to exposure to the stressor to evaluate changes in nociceptive thresholds induced by pharmacological manipulations. Ceiling tail-flick latencies were 10s except where noted. Tail-flick latencies, measured at baseline or prior to administration of the stressor, did not differ between groups in any study.

### Data Analyses.

Results were analyzed using ANOVA, repeated measures ANOVA and Fisher's PLSD post hoc tests. P<0.05 was considered significant.

### Supplemental Methods.

CB₁ and CB₂ binding assays were conducted in rat cerebellar membranes and CB₂-overexpressing CHO cells (Receptor Biology-Perkin Elmer, Wellesley, Mass.), respectively, using [³H]WIN-55212-2 (NEN-Dupont, Boston, Mass., 40-60 Ci/mmol) as a ligand. We measured phospholipase C and phospholipase D activities at 37° C. for 15 min in 35 mM Tris-maleate buffer (0.5 ml, pH 7.3) containing calcium chloride (5 mM), fatty acid-free BSA (2 mg-ml-1, Sigma), phospholipase C (B. cereus, 1 U; Sigma) or phospholipase D (S. chromofuscus, 10 U, Sigma). Phosphatidylcholine [³H]methylcholine (8 mM, ARC, 60 ci/mmol, 20,000 dpm) was used as a substrate. Reactions were terminated by adding chloroform:methanol (1:1, 1 ml). Radioactivity was determined by liquid scintillation counting. DGL activity was measured at 37° C. for 30 min in 0.5 ml Tris buffer (50 mM, pH 7.5), rat brain protein (800 g, supernatant, 100 mg protein) and [³H]dioleoylglycerol (50 μM, 75,000 dpm; ARC, St. Louis, Mo.). After stopping the reactions with chloroform/methanol (1:1, 1 ml), we collected 0.5 ml of organic layer and added 5 μg of diolein, 5 μg monoleoylglycerol and 12.5 μg oleic acid and dried under a stream of nitrogen. Thin-layer chromatography analyses were carried out on silica gel G plates, eluted with a solvent system consisting of chloroform/methanol/ammonium hydroxide (85:15:0.1). Lipids were visualized by iodine staining, and the bands scraped. Radioactivity was determined by liquid scintillation counting. We performed cyclooxygenase (Cox) assays with a commercial kit using purified enzymes (Cox-1 from ram seminal vesicles, Cox-2 human recombinant) (Cayman Chemicals, Ann Arbor, Mich.).

Each publication, patent application, patent, and other reference cited herein is incorporated by reference in its entirety to the extent that it is not inconsistent with the present disclosure. In particular, all publications cited herein are incorporated herein by reference in their entirety for the purpose of describing and disclosing the methodologies, reagents, and tools reported in the publications that might be used in connection with the invention. Nothing herein is to be construed as an admission that the invention is not entitled to antedate such disclosure by virtue of prior invention.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A method of potentiating stress-induced analgesia in a mammalian subject in need thereof comprising administering to the subject at least one compound selected from the group consisting of: 2-arachidonylglycerol hydrolysis inhibitors, analgesics, opioids, NSAIDs, FAAH inhibitors, PPARα agonists, anandamide transport inhibitors, CB1 receptor agonists, and anxiolytics.

2. A method for treating a stress-induced disorder or condition in a mammalian subject in need thereof, comprising administering to the subject a therapeutically effective amount of at least one compound selected from the group consisting of: 2-arachidonylglycerol hydrolysis inhibitors, analgesics, opioids, NSAIDs, FAAH inhibitors, PPARα agonists, anandamide transport inhibitors, CB1 receptor agonists, anxiolytics, and antidepressants.

3. The method of claim 2, wherein the subject is human.

4. A method for enhancing or potentiating stress-induced analgesia in a mammalian subject in need thereof, comprising administering at least one compound that stimulates central nervous system cannabinoid receptors.

5. The method of claim 4, wherein the compound is selected from the group consisting of: 2-arachidonylglycerol hydrolysis inhibitors, FAAH inhibitors, anandamide transport inhibitors, and CB1 receptor agonists.

6. A method of producing analgesia in a patient in need thereof, wherein the patient is tolerant to morphine, comprising stimulation of central nervous system cannabinoid receptors.

7. The method of claim 6, wherein the stimulation of central nervous system cannabinoid receptors is the result of administration of at least one compound selected from the group consisting of: 2-arachidonylglycerol hydrolysis inhibitors, FAAH inhibitors, anandamide transport inhibitors, and CB1 receptor agonists.

8. A method of producing analgesia in a patient in need thereof, wherein the patient is tolerant to morphine, comprising administration of at least one compound selected from the group consisting of: 2-arachidonylglycerol hydrolysis inhibitors, FAAH inhibitors, anandamide transport inhibitors and CB1 receptor agonists.

9. A method of testing or screening compounds that mediate non-opioid stress-induced analgesia comprising,
a) inducing sufficient stress in an animal to produce analgesia,
b) administering the compound at a dose sufficient to stimulate central nervous system cannabinoid receptors;
c) measuring the amount of analgesia produced following administration; and
d) comparing the amount of analgesia before and after administration of the compound.

10. The method of claim 9, wherein the drug is administered directly into the central nervous system.

11. The method of claim 9, wherein the animal is rendered tolerant to opioids prior to testing.

12. The method of claim 9, wherein the compound is an antagonist of endocannabinoid receptors.

13. The method of claim 9, wherein the compound blocks an enzyme that mediates the inactivation of endogenous cannabinoids.
